(19) United States
(12) Patent Application Publication
(10) Pub. No.: US 2022/0054018 A1
(43) Pub. Date: Feb. 24, 2022

(54) MEASURING DEVICE FOR MEASURING AN INTENSIVE MEASURAND

(71) Applicant: COURAGE + KHAZAKA ELECTRONIC GMBH, Köln (DE)

(72) Inventors: Michael HOSS, Weilerswist (DE); Georg WIORA, Köln (DE)

(73) Assignee: COURAGE + KHAZAKA ELECTRONIC GMBH, Köln (DE)

(21) Appl. No.: 17/599,711

(22) PCT Filed: Mar. 27, 2020
(86) PCT No.: PCT/EP2020/058816

§ 371 Date: Sep. 29, 2021

(30) Foreign Application Priority Data
Mar. 29, 2019 (DE) 10 2019 204 511.1

(51) Int. Cl. A61B 5/01; G01N 13/00

(52) U.S. Cl. CPC A61B 5/01; G01N 13/00; G01N 2013/003; A61B 2562/029; A61B 2562/06; A61B 2562/0271

(57) ABSTRACT

In a measuring device for measuring an intensive measurand, including at least one measuring chamber having at least one opening, the opening being placeable on the body to be examined. At least three sensors for measuring the intensive measurand are arranged in the measuring chamber, the sensors being arranged at different distances from the body to be examined during measurement. An evaluation device being provided which receives the values measured by the sensors and determines a total value for the intensive measured variable from the at least three measured values as well as a substance or energy diffusion rate.

The invention relates to a measuring device for measuring an intensive measurand, in particular the concentration of a substance emitted or absorbed by a body by diffusion or the temperature according to the pre-characterizing part of claim 1 as well as a method for measuring an intensive measurand, in particular the concentration of a substance emitted or absorbed by a body by diffusion or the temperature according to claim 16.

For the evaluation of bodies, in particular technical or biological membranes, the diffusion behavior of the bodies is interesting, for example. A body can be a biological membrane, for example. A biological membrane can be the skin surface, for example. In the current prior art, an intensive measurand, namely the concentration of a substance emitted by a body by diffusion, can be measured. From this, the diffusion rate J of the substance through the membrane can be determined and this can be used as a measure for the diffusion behavior of the membrane. Devices such as those described in DE 2553377 are used for this purpose. Such devices comprise at least one measuring chamber having two openings, one of the at least two openings being placeable on the membrane to be examined. Two sensors are arranged in the measuring chamber, the sensors being arranged at different distances from the body to be examined during measurement. In this way, the diffusion rate can be determined.

In the prior art known to date, measuring chambers with only one opening and only one sensor are also known. A freezing plate will then be provided on the side opposite the first opening to serve as a diffusion sink.

However, there is an increasing need for this measurement to be faster and more reliable.

Therefore, it is an object of the present invention to provide a measuring device and a method for measuring an intensive measurand in which the measuring is faster and more reliable.

This object is achieved by the features of claims 1 and 16.

The invention advantageously provides that in a measuring device for measuring an intensive measurand, in particular the concentration of a substance emitted by a body by diffusion or the temperature, comprising at least one measuring chamber having at least one opening, the opening being placeable on the body to be examined, that in the measuring chamber at least three sensors for measuring the intensive measurand are arranged, the sensors being arranged at different distances from the body to be examined during measurement, an evaluating device being provided which receives the values measured by the sensors and determines a total value for the intensive measurand from the values measured at least at three different distances from the body.

The intensive measurand is a state variable that does not change with varying size of the system under consideration. In the present case, an intensive measurand may be the concentration of a substance emitted by a body by diffusion or the temperature. It can also be the pressure, electrical voltage or all kinds of concentration or density. The intensive measurand can be directly or indirectly measured.

The measuring device comprises at least one measuring chamber having at least one opening, the opening being placeable on the body to be examined.

It is particularly preferred that the measuring chamber has at least two opening, at least one of the two openings being placeable on the body to be examined. The measuring chamber having at least two openings thus forms an open measuring chamber.

Alternatively, the measuring chamber can have only one opening being placeable on the body to be examined, and a freezing plate can be provided on the side opposite the opening, which serves as a diffusion sink. Such a measuring chamber would be a closed measuring chamber.

The present invention has the advantage of having several spatially resolved values, with the associated sensors having different distances from the membrane, and more accurate results can be obtained by evaluating all values. The measurement results are also available more quickly.

A calculation rule is preferably stored in the evaluating device, on the basis of which the evaluating device determines the total value.

At the start of a measurement, when the device is placed on the skin, for example, it takes a certain amount of time for the substances emitted by diffusion from the body to reach the individual sensors, for example. Therefore, the sensors cannot yet measure the corresponding value immediately and it takes some time until the measured value is stable. Such processes can be taken into account in a stored calculation rule.

As a calculation rule, for example, a model function can be stored in the evaluating device or in a downstream separate evaluating unit for the approximate simulation of the real course of an intensive measurand to be determined.

After the measuring device has been placed on the body to be examined, for example, the temperature measurement value determined by the sensors only slowly adjusts from an initial measured value (Tᵢₙᵢₜᵢₐₗ) to 1 the real body temperature value as the final convergence temperature (T_{final}) of the sensors, so that a certain compensation period is required to measure the real body temperature so that the sensors of the measuring device have adjusted to the real body temperature or final convergence temperature (T_{final}). The compensation period of the measuring device can be up to 300 seconds.

According to the invention, it can be provided to simulate a model function for determining the real body temperature or the final convergence temperature (T_{final}) in order to simulate the temporal course of the temperature measurement values after the measuring device has been placed on the body surface, starting from an initial temperature (Tᵢₙᵢₜᵢₐₗ) the real body temperature or the final convergence to temperature (T_{final}). The model function makes it possible to calculate the final body temperature value or the final convergence temperature (T_{final}) by means of measured values recorded during a short measuring period after the measuring device has been placed on the body while the temperature is still equalizing. In particular, an exponential function can be stored as a model function in the evaluating device or a downstream separate evaluating unit, for example for the temperature course.

Particularly preferably, the following function is stored as a model function for the temperature course of the sensors of the measuring device in the evaluating device or a downstream separate evaluating unit:

T(t,T₀,T₁,τ)=T₀+(T₁−T₀)·(1−e^{-t·τ}); with

T₀=temperature at the point in time t=0, T₁ as final convergence temperature with t=∞, τ=time constant of exponential function in [1/s], t as point in time or time period in [s] and T as current temperature at the point in time t.

During a defined time period after the measuring device has been placed on the body to be measured, temperature measurement values Tₜ_{ᵢ} are determined by the sensors at defined times tᵢ. Preferably, a minimum measurement period of 20 to 30 seconds is selected for determining the measured values. It is particularly preferred that the start of the measurement period is selected starting from a time period in the range of 10 to 20 seconds after placing the measuring device on the body.

The time constant τ of the model function can be considered as an unknown of the model function or, alternatively, can be determined for the measuring device by measuring real temperature courses and stored in the evaluating device. Particularly preferably, τ can be set to a value in the range of 1/60 to 1/90 [1/s].

In general, the variable T₀ is the temperature at the point in tme t=0 for the assumed model function or the course of the temperature over the time period t approximated by the model function, which is to be determined as an unknown.

However, the temperature T₀ can be set to the initial temperature (Tᵢₙᵢₜᵢₐₗ) determined via the measuring device or to any intermediate temperature from which the temperature course is to be approximated via the model function.

The final convergence temperature T₁ at the point in time t=∞ corresponds to the real body temperature (T_{final}) with a theoretically assumed best possible approximation of the temperature course via the model function.

The total value of the intensive measured value can be used as the measured value T for the current temperature measured at the point in time t, which in turn can be calculated from the measured values of the sensors using a calculation rule.

The two unknowns of the model function T₀ and T₁ or alternatively the three unknowns of the model function τ, T₀ and T₁ can be determined by using a Levenberg-Marquardt algorithm or approximated from the pair of measured values.

The Levenberg-Marquardt algorithm can be combined in an iterative procedure with robust statistical methods (maximum-likelihood estimators) to sort out measurement points whose values do not fit the model function on the basis of the distribution function of the residuals. An improved estimate of the model parameters can then be made on the reduced set of measurement points.

The body to be examined can be any diffusion source or sink. Preferably, the body can be a technical or biological membrane. A biological membrane can be the skin surface, for example.

By means of the sensors, for example, the concentration c of the substance emitted by the body by diffusion can be measured directly or indirectly. The concentration gradient ∇c can be calculated from the spatial concentration distribution c(z). From this, the diffusion rate J of the corresponding substance emitted by diffusion can be calculated according to Fick's law with the aid of the substance pair-specific diffusion constant D:

J=−D·∇c(z)

The diffusion constant is known for certain substance pairings and can be looked up in the literature. Alternatively, the diffusion constant can also be determined experimentally. The diffusion constant depends on the pressure and the temperature. However, the diffusion constant is known for certain pressures and temperatures. In principle, the concentration gradient can be determined from the measurement of c(z) at two points z₁ and z₂. For this purpose, the value of the gradient has to be estimated from the two measured values. This is possible, for example, with the following linear difference approach:

$$J = - D \cdot \frac{c ( z 2 ) - c ( {z}_{1} )}{{z}_{2} - {z}_{1}}$$

However, a spatially higher resolved concentration measurement allows the gradient of the concentration to be derived from the measured values with much higher reliability. If one determines the concentration c(zᵢ) at n distances z₁ to zₙ, the measuring points can be regarded as interpolation points of an arbitrary parameterizable function. For example, this can be polynomial p(z) of degree k.

$${p}_{k} ( z ) = {\sum }_{j = 0}^{k - 1} {a}_{j} \cdot {z}^{j}$$

By known numerical methods, the polynomial parameters a₀ to aₖ₋₁ can be determined such that the following applies:

$$E ( {a}_{0} , … , {a}_{k - 1} ) = {\sum }_{i = 0}^{n - 1} {( {p}_{k} ( {z}_{i} ) - c ( {z}_{i} ) )}^{2} ⇒ min .$$

This gives the possibility to calculate the analytical derivative ∂c/∂z of the concentration:

$$∇ c ( z ) = \frac{\partial c}{\partial z}$$

If p is a function whose gradient is not constant, it can be used to determine a location-dependent gradient. Since the target variable to be determined is the diffusion rate J, both the determination of the parameters of p and the calculation of ∇c(z) can be chosen so that the time course of J, for example, is as stable and robust as possible against disturbances or responds as quickly as possible after the measuring device has been placed on the surface.

The substance emitted by the body by diffusion can be water vapor. If the body if the skin, it is called transepidermal water loss. The diffusion rate of water vapor through the skin is determined as the measurand for this purpose. Such a measuring value is called TEWL value.

The calculation rule stored in the evaluating device can weight the values measured by the sensors differently to determine the total value for the intensive measurand.

This has the advantage that more accurate results are available much faster. A sensor placed closer to the body can measure the corresponding values faster than a sensor placed further away, because when the device is placed on the body again, it takes a certain time for the amount of substance emitted by diffusion to reach the corresponding sensors.

For each device test measurements can be performed and for special devices can be stored at which time of a measurement which values are weighted how in order to achieve the best values.

The calculation rule stored in the evaluating device can use a linear estimator, a non-linear estimator or a robust estimator when determining the total value. A plurality of robust estimators are known.

Such a robust estimator has the advantage that the values that deviate substantially are not taken into account. In this way, more accurate results can be obtained.

The total value is a value determined from all measured values. This value is supposed to represent the actual intensive measurand. If the measurement is performed for a long time, then the measured value will be very stable for all sensors and the total value could be, for example, an average value of all measured values. However, depending on the application of the device, there may be different calculation rules and, for example, at the start of a measurement, different weightings of the measured values of the sensors may be applied, as already described above. Even if, for example, individual values varying strongly due to air turbulence in the environment, this can be recorded and taken into account. Robust estimators, for example, cannot take into account these widely varying values.

The total value can be an estimated value which the evaluating device determines on the basis of the calculation rule, wherein the calculation rule takes into account a temporal course of the total value.

Through test trials, for example, the temporal course of the total value over time can be known. If a new measurement is now started and the device is placed on the body and the first measurements are available, an estimated value for the total value can be determined on the basis of a stored typical temporal course of the total value.

The sensors can be arranged in the center of the measuring chamber or in the sidewall.

The measuring chamber can comprise at least one sidewall and the at least three sensors can be arranged at the at least one sidewall at different distances from the body to be examined. Alternatively, the sensors can be placed in the center or in the central area of the measuring chamber.

The at least one sidewall can be arranged between the first and the second opening.

The measuring chamber can have a round cross-section.

The at least three sensors can be arranged in at least three rows, the at least three rows being arranged at different distances from the body to be examined and at least one sensor being arranged per row.

At least five sensors can also be provided. Thus, at least five rows can be provided, wherein several sensors can be provided per row. For example, six sensors can be arranged per row so that a total of at least thirty sensors can be provided.

The sensors can measure the concentration of a substance emitted by the body by diffusion. The sensors measuring the concentration of a substance emitted by diffusion can additionally measure the temperature and/or relative humidity. Alternatively, in addition to the sensors measuring the concentration of a substance emitted by diffusion, at least three temperature sensors and/or sensors for measuring relative humidity for measuring the temperature and/or relative humidity can be provided which are also arranged at different distances from the body to be examined during measurement.

The evaluating device can also receive the measured values for the temperature and/or relative humidity and determine a total temperature value and/or total value for relative humidity based on the measured values.

The additional temperature sensors and/or sensors for relative humidity can also be arranged on the sidewall or in the central area or in the center of the measuring chamber.

According to the present invention, a method for measuring an intensive measurand, in particular the concentration of a substance emitted by a body by diffusion or the temperature, can be provided, the method comprising the following steps:
placing at least one measuring device having at least three sensors for measuring the intensive measurand on a body to be examined, the measuring chamber having at least one opening that is placed on the body to be examined,
wherein the measuring chamber is placed such that the sensors are arranged at different distance from the body to be examined during measurement,
wherein the evaluating device receives the values measured by the sensors, and a total value for the intensive measurand is determined by the evaluating device from the measured values.

To determine the total value, a calculation rule can be stored, which is used to determine the total value.

When determining the total value, the values of the calculation rule measured by the sensors can be differently weighted.

The measured values of the sensors that are arranged closer to the membrane to be examined can be weighted higher.

Due to the different weighting, a more reliable value can be determined more quickly.

A robust estimator can be used to determine the total value.

It is also possible to determine a value for the intensive measurand for a specific distance from the surface that was not measured directly by means of a sensor.

The presence of different measured values from sensors located at different distances from the body to be examined allows a function to be determined that represents the dependence of the values in relation to the distance from the membrane. In this way, values can also be determined for specific distances that are not directly determined by means of a sensor. In this way, the intensive measurand can also be determined directly on the body surface. Provided that the temperature or relative humidity is measured as an intensive measurand with the sensors or with additional sensors, the temperature or relative humidity on the body surface can be determined.

The concentration of a substance emitted by diffusion, the temperature or the relative humidity in the immediate environment outside a second opening of the sensor can additionally be indicated.

Additionally, the temperature can be measured at at least three points that are at different distances from the body.

In the following, exemplary embodiments of the invention are described in more detail with reference to the drawings, in which the following is schematically shown:

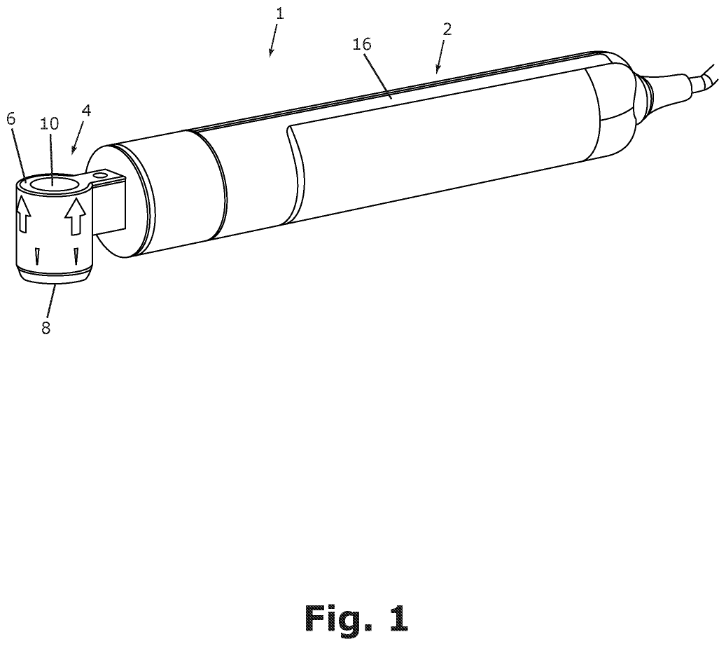

FIG. 1 shows the measuring device for measuring the amount of a substance emitted by a body by diffusion,

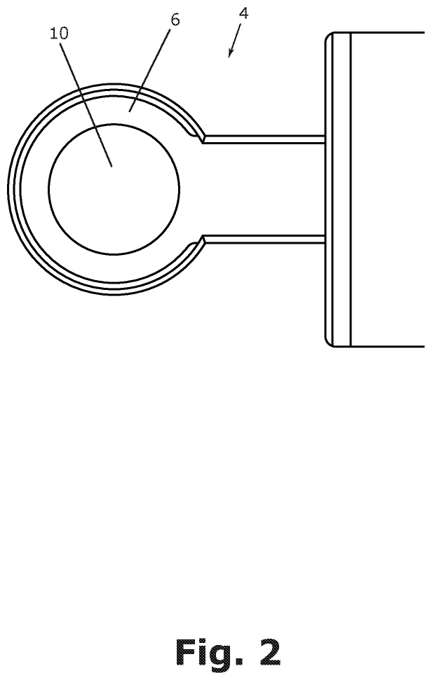

FIG. 2 shows a plan view of the measuring chamber,

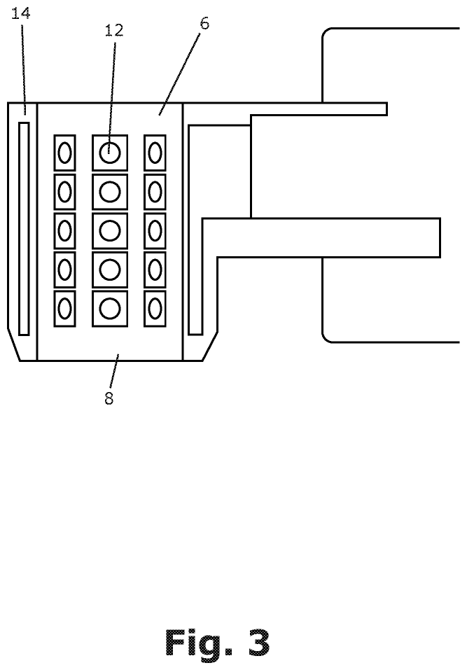

FIG. 3 shows a section through the measuring chamber,

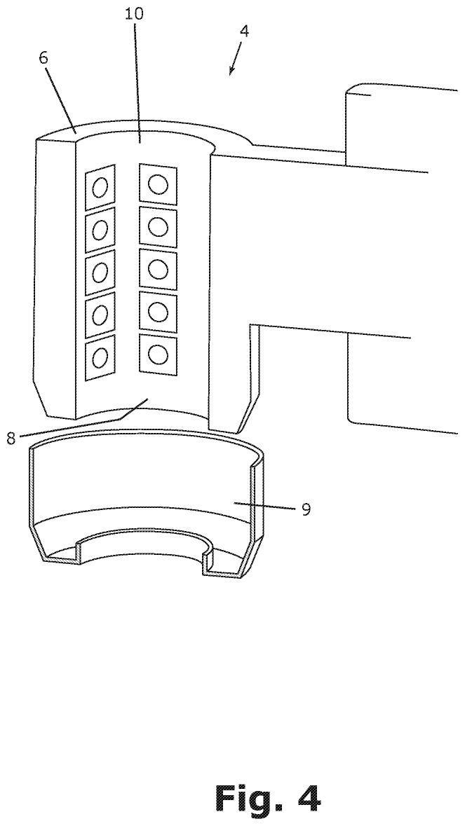

FIG. 4 also shows a section through the measuring chamber and a section through a touchdown cap,

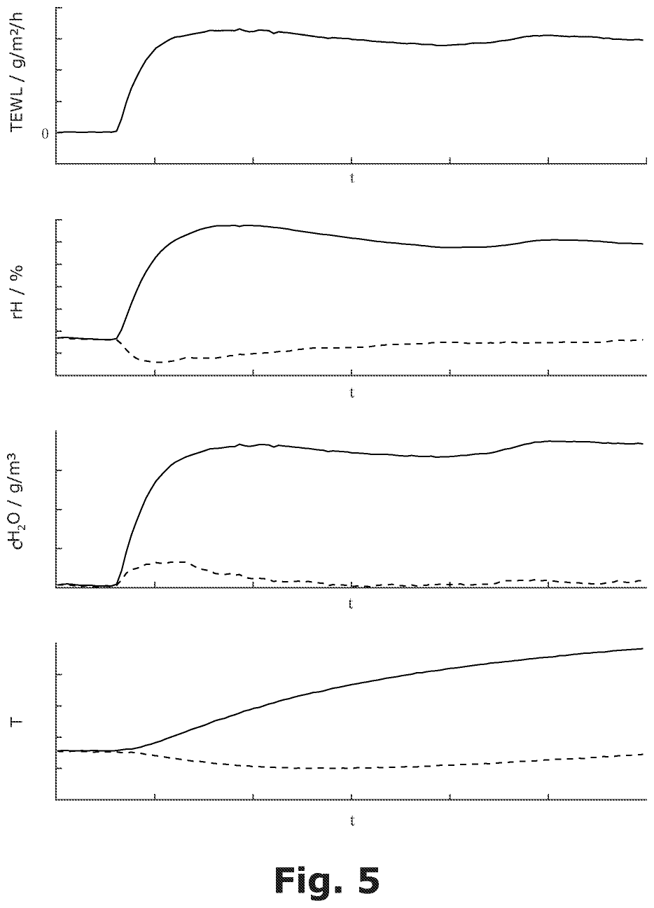

FIG. 5 shows the temporal course of the measurements and the extrapolation of water vapor concentration, temperature and relative humidity to the surface of the body (continuous curve) and to the second opening of the measuring device (dashed curve).

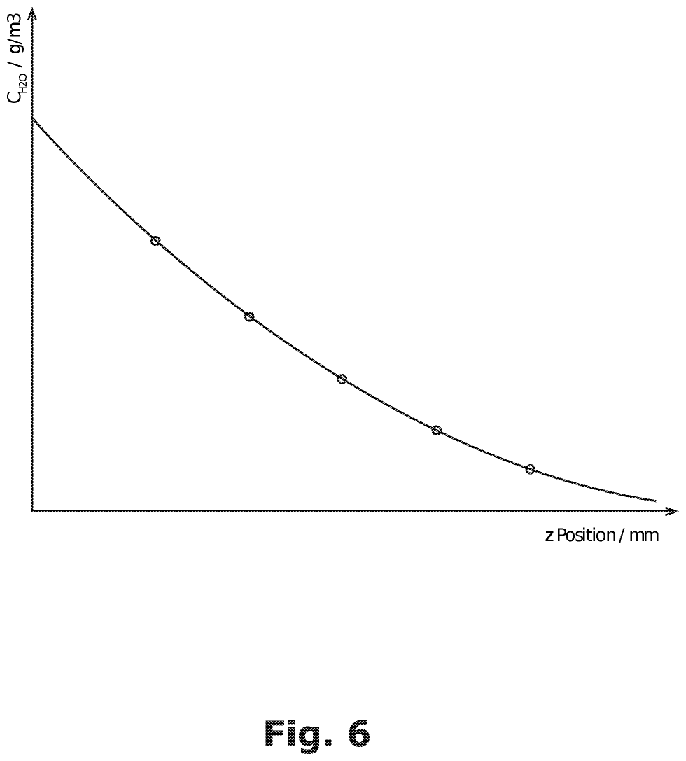

FIG. 6 shows the extrapolation of the water vapor concentration as a function of the position of the sensor to the body.

FIG. 1 shows the measuring Device for measuring an intensive measurand. In the present exemplary embodiment, the concentration of a substance emitted by a body 5 by diffusion is measured.

A handle 2 is shown. A head 4 with a measuring chamber 6 is arranged on the handle. In the illustrated exemplary embodiment, measuring chamber 6 has at least two opening 8 and 10, one of the at least two opening 14 being placeable on a body 5 to be examined. In the present case, opening 8 can be placed on the body 5 to be examined. The measuring chamber 6 is shown in plan view in FIG. 2. The measuring chamber 6 has a round cross-section as can be seen in FIG. 2.

FIG. 3 shows a section through measuring chamber 6. It can be seen that a plurality of sensors 12 are arranged on sidewall 14 of measuring chamber 6. The sensors 12 are arranged in rows and columns next to or on top of each other. Five sensors are arranged in a row on top of each other. Six sensors are arranged in a row, with an average of three sensors 12 of a row being visible.

The sensors 12 directly or indirectly measure the concentration of a substance emitted by diffusion.

The body 5 can be a biological or technical membrane. The measuring chamber 6 can be placed on body 5. A biological membrane can be a skin surface, in particular.

The illustrated sensors 12 can additionally also measure the temperature as intensive measurand. Alternatively, separate sensors can also be provided which measure the temperature, wherein a plurality of sensors can also be provided to measure the temperature.

An evaluating device 16 is arranged in handle 2 or externally.

The evaluating device 16 receives the values measured by the sensors 12 and determines a total value for the concentration of the substance emitted by diffusion from the at least three measured values. Since thirty sensors are provided in the present case, the measured values of at least thirty sensors are provided. A calculation rule is preferably stored in evaluating device 16, on the basis of which evaluating device 16 determines the total value.

The calculation rule and thus evaluating device 16 can differently weight the values measured by the different sensors 12. For example, the values of those sensors 12 that are arranged closer to the body to be examined can be weighted higher. Said sensors 12 are less susceptible to interference due to air turbulences. Furthermore, with these sensors 12, the measured values are available more quickly after measuring chamber 6 has been placed on a body again. The substance emitted by diffusion must first reach the sensors 12 after placing the measuring device 1 on the body. Therefore, said sensors 12 can measure the substance emitted by diffusion only after a certain time.

FIG. 4 also shows a section through the device, wherein an additional touchdown cap is illustrated. Said touchdown cap is used to protect the skin surface, in particular during examinations of the skin surface.

FIG. 5 shows the temporal course of measurements and the extrapolation of water vapor concentration, temperature and relative humidity to the surface of the body (continuous curve) and to the second opening of the measuring device (dashed curve). A stable value is reached only after a certain time. However, an estimated value for the total value can also be determined on the basis of the measured values that have already been measured at an early stage. To determine the estimated value, evaluation device 16, and thus the calculation rule, takes into account the temporal course of the total value and/or the measured values. The temporal course can be determined by comparative tests and, for example, a typical temporal course function can be determined. If the first values are now available, the expected stable total value can be determined on the basis of these first values and the stored temporal course function. Even if a measurement lasts longer, air turbulences or other disturbances may occur.

When determining the total value, measured values that vary strongly from the other measured values cannot be taken into account.

Thus, different weightings can still be applied in the further course of the measurement.

For example, the calculation rule may use a robust estimator to determine the total value.

It is also possible to determine the concentration of a substance emitted by diffusion at the body surface.

It is also possible to determine the concentration of a substance emitted by diffusion in the immediate vicinity of the measuring device.

For this purpose, an extrapolation can be performed, for example, as shown in FIG. 6.

FIG. 6 shows the water vapor concentration as a function of the distance between the sensors and the membrane to be examined. A function can be determined on the basis of the measured values. By determining this function, conclusions can be drawn about what the water vapor concentration is at the body surface and in the environment.

1-27. (canceled)

28. A measuring device for measuring an intensive measurand, in particular the concentration of a substance emitted by a body by diffusion or the temperature, comprising at least one measuring chamber having at least one opening, the opening being placeable on the body to be examined,
wherein at least three sensors for measuring the intensive measurand are arranged in the measuring chamber, the sensors being arranged at different distances from the body to be examined during measurement,
wherein an evaluating device is provided that receives the values measured by the sensors and determines a total value for the intensive measurand from the at least three measured values.

29. The measuring device according to claim 28, wherein the measuring chamber comprises at least two openings.

30. The measuring device according to claim 28, wherein a calculation rule is stored in the evaluating device, on the basis of which the evaluating device determines the total value.

31. The measuring device according to claim 30, wherein the calculation rule stored in the evaluating device weights the values measured by the sensors differently to determine the total value for the intensive measurand.

32. The measuring device according to claim 30, wherein the calculation rule stored in the evaluating device uses a robust estimator when determining the total value.

33. The measuring device according to claim 30, wherein the total value is an estimated value which the evaluating device determines on the basis of the calculation rule, wherein the calculation rule takes into account a temporal course of the total value.

34. The measuring device according to claim 28, wherein a model function is stored in the evaluating device or in a downstream separate evaluating unit for the approximate simulation of the real course of an intensive measurand to be determined.

35. The measuring device according to claim 28, wherein the measuring chamber comprises at least one sidewall and the at least three sensors are arranged on the at least one sidewall at different distances from the body to be examined.

36. The measuring device according to claim 28, wherein the measuring chamber comprises at least one sidewall and the at least three sensors are arranged spaced from the sidewall in the central area of the measuring chamber at different distances from the body to be examined.

37. The measuring device according to claim 28, wherein the measuring chamber has a round cross-section.

38. The measuring device according to claim 28, wherein the at least three sensors are arranged in at least three rows, the at least three rows being arranged at different distances from the body to be examined and at least one sensor being arranged per row.

39. The measuring device according to claim 38, wherein the sensors measure the concentration of a substance emitted by the body by diffusion.

40. The measuring device according to claim 39, wherein the sensors measuring the concentration of a substance emitted by diffusion additionally measure the temperature and/or relative humidity, or that additionally at least three temperature sensors and/or sensors for measuring relative humidity for measuring the temperature and/or relative humidity are provided which are also arranged at different distances from the body to be examined during measurement.

41. The measuring device according to claim 40, wherein the evaluating device also receives the measured values for the temperature and/or relative humidity and determines a total temperature value and/or total value for relative humidity based on said measured values.

42. The measuring device according to claim 40, wherein the temperature sensors and/or sensors for relative humidity are also arranged on the sidewall.

43. A method for measuring an intensive measurand, in particular the concentration of a substance emitted by a body by diffusion or the temperature, comprising:
placing at least one measuring chamber having at least three sensors for measuring the intensive measurand on a body to be examined, the measuring chamber having at least one opening that is placed on the body to be examined,
wherein the measuring chamber is placed such that the sensors are arranged at different distance from the body to be examined during measurement,
wherein the evaluating device receives the values measured by the sensors, and a total value for the intensive measurand is determined by the evaluating device from the measured values.

44. The method according to claim 43, wherein a calculation rule is stored for determining the total value, on the basis on which the total value is determined.

45. The method according to claim 44, wherein when determining the total value, the values measured by the sensors are weighted differently in the calculation rule.

46. The method according to claim 45, wherein the measured values of the sensors that are arranged closer to the body to be examined are weighted higher.

47. The method according to claim 43, wherein a robust estimator is used for determining the total value.

48. The method according to claim 43, wherein a model function is used for approximate simulation of the real course of an intensive measurand to be determined.

49. The method according to claim 48, wherein a value for the intensive measurand for a specific distance from the surface that was not measured directly by means of a sensor can be determined.

50. The method according to claim 39, wherein the concentration of a substance emitted by a body by diffusion is measured as intensive measurand.

51. The method according to claim 47, wherein, additionally, the temperature and/or relative humidity is measured as an intensive measurand at at least three points which are at a different distance from the body.

52. The method according to claim 43, wherein it determines the diffusion rate for the corresponding measurand from the gradient ∇c(z) of the measured intensive measurand c(z) in analogy to Fick's law.

53. The method according to claim 52, wherein the intensive measurand is the temperature and the diffusion rate is the heat loss.

54. The method according to claim 52, wherein the intensive measurand is a substance concentration and the diffusion rate is a substance quantity per time and per area.
